(19) United States
(12) Patent Application Publication
(10) Pub. No.: US 2004/0147442 A1
(43) Pub. Date: Jul. 29, 2004

(54) Thrombin-inhibiting peptides

(76) Inventors: Christiane Noeske-Jungblut, Berlin (DE); Ursula Egner, Berlin (DE); Peter Donner, Berlin (DE); Wolf-Dieter Schleuning, Berlin (DE); Wolfram Bode, Gauting (DE); Pablo Fuentes Prior, Martinsried (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/694,847

(22) Filed: Oct. 29, 2003

Related U.S. Application Data
(62) Division of application No. 09/445,214, filed on May 3, 2000, now abandoned.
Application No. 09/445,214 is a 371 of international application No. PCT/EP98/03356, filed on Jun. 5, 1998.

(30) Foreign Application Priority Data
Jun. 6, 1997 (DE) 197 24 791.1

(51) Int. Cl. C12Q 1/68; A61K 38/16

(52) U.S. Cl. 514/12; 530/324

(57) ABSTRACT

Peptides of Formula I are useful for therapeutic purposes, among others.

This invention relates to peptides and their use for the production of pharmaceutical agents.

The protease thrombin has a key role in blood clotting. It cleaves fibrinogen into fibrin, which then forms a blood clot. Under physiological conditions, this results in the stopping of bleeding and the closure of the wound. Under pathological conditions, however, if, e.g., vascular lesions based on arteriosclerotic changes or produced by a myocardial infarction are present, it can result in a complete occlusion of the vessel. This manifests itself in, e.g., the occurrence of thromboses or a myocardial infarction. Therefore, thrombin inhibitors are used for the treatment of thromboses.

A known thrombin inhibitor is the protein hirudin, which was originally obtained from leeches (Markwardt, F. (1957) Z. Physiol. Chem. 308, 147-156). Its three-dimensional structure in the complex with thrombin is known (Rydel, T. J. et al. (1990) Science 249, 277-280). An equally effective inhibitor is the triabin that is isolated from a hematophagous bug (Noeske-Jungblut, C. et al (1995) J. Biol. Chem. 270, 28629-28634). These two inhibitors differ in their mode of action. While the hirudin binds to two points of the thrombin, the active center and a so-called anion binding site, the triabin binds only to the anion binding site. In this case, the active center is not blocked; it is, however, inhibited, despite the fibrinogen cleavage, since the binding of the fibrinogen to this anion binding site is necessary for the cleavage. A peptide that is derived from the hirudin is the hirulog, which just like hirudin blocks the active center and the anion binding site of the thrombin (Maraganore, J. M. and Bourdon, P. (1990) Biochemistry 29, 7095-7101). It is about 100 times less effective than the hirudin (J. M. Maraganore et al. 1990, Biochemistry 29, 7095-7101).

In clinical studies, it has been shown that hirudin can be easily overdosed (e.g., Studie TIMI 9A, Antman, E. M. (1994) Circulation 90, 1624-1630 or Studie Gusto IIa (1994) Circulation 90, 1631-1637) and then results in severe bleeding complications. Pre-clinical data show that triabin has another inhibiting kinetics. Just like hirudin, it inhibits at low concentrations, but does not show any complete inhibition of clotting at high concentrations (see sample application 1). For clinical use, this would mean that triabin can be used in a broader dose range, without resulting in bleeding that is too severe.

The disadvantage of triabin is that it is a relatively large protein and therefore must be administered intravenously. Smaller peptides, which have the same properties as triabin, would therefore have the advantage that they can also be administered orally or transdermally. Further advantages of smaller peptides consist in the fact that they can be produced more simply and thus are less expensive. Other advantages relative to large proteins then consist in the fact that smaller peptides have better storage properties.

Peptides of general formula I have now been found

| Y¹-X¹-Ser-X²-Ser-X³-X⁴-Asn-Phe-X⁵-X⁶-X⁷-Y²-D-Tyr-X⁸-Val-X⁹-Glu-X¹⁰- | (I) |
|---|---|
| 1 2 3 4 5 6 7 8 9 10 11 12 13 d 14 15 16 17 18 19 | |
| X¹¹-X¹²-Ser-X¹³-X¹⁴-Asp, | |
| 20 21 22 23 24 25 | |

in which

Y¹ is Phe, Lys, Cys and Orn, and

Y² is Asp, Cys and Glu, and Y¹ also has the meaning of Y², and Y² has the meaning of Y¹, whereby Y¹ and Y² are linked to one another via a side chain or a β-turn mimetic agent, and

X¹⁻¹⁴ represents any amino acid, which can be connected to one another via side chains, which have better properties compared to the known peptides.

Preferred peptides of general formula I are those in which

Y¹ is Phe, Lys, Cys and Orn, and

Y² is Asp, Cys and Glu, and Y¹ also has the meaning of Y², and Y² has the meaning of Y¹, whereby Y¹ and Y² are linked to one another via a side chain or a β-turn mimetic agent, and

X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn, Cit. β-Ala, homo-Cys, homo-Ser, Gaba, Can, β-CN-Ala, OH-Pro, OH-Lys, N-Met-Lys, Met-His, desmosine and djenkolic acid, which can be connected to one another via side chains.

Especially preferred peptides of general formula I are those in which

Y¹ is Phe, Lys, Cys, and Orn, and

Y² is Asp, Cys, and Glu, and Y¹ also has the meaning of Y², and Y² has the meaning of Y¹, whereby Y¹ and Y² are linked to one another via a side chain or a β-turn mimetic agent, and

X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn, and Cit, which can be linked to one another via side chains.

Especially preferred are those peptides of general formula I,

in which

Y¹ is Lys, Cys and Orn, and

Y² is Asp, Cys, and Glu, and Y¹ also has the meaning of Y², and Y² has the meaning of Y¹, whereby Y¹ and Y² are linked to one another via a side chain, and

X⁶ and X⁸ are Leu,

X⁷ is Val and

X¹⁻⁵ and X⁹⁻¹⁴ are Ala, Val, Leu, Ile, Pro, Phe, Trp, Met, Gly, Ser, Thr, Cys, Tyr, Asn, Gln, Asp, Glu, Lys, Arg, His, Orn and Cit, whereby if

X⁴ stands for Glu, and X¹⁰ stands for Lys, the latter are linked to one another via a side chain.

In particular, those peptides of general formula I are also preferred,

in which

Y¹ is Lys, and

Y² is Asp, and Y¹ also has the meaning of Y², and Y² has the meaning of Y¹, whereby Y¹ and Y² are linked to on another via a β-turn mimetic agent, and

X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn and Cit, whereby, if X⁴ stands for Glu and X¹⁰ stands for Lys, the latter are linked to one another via a side chain.

Those peptides of general formula I thereof are also especially preferred,

in which

Y¹ is Lys, and

Y² is Asp, and Y¹ also has the meaning of Y², and Y² has the meaning of Y¹, whereby Y¹ and Y² are linked to one another via a β-turn mimetic agent, and

X⁶ and X⁸ are Leu,

X⁷ is Val,

X¹⁻⁵ and X⁹⁻¹⁴ are Ala, Val, Leu, Ile, Pro, Phe, Trp, Met, Gly, Ser, Thr, Cys, Tyr, Asn, Gln, Asp, Glu, Lys, Arg, His, Orn and Cit, whereby if

X⁴ stands for Glu and X¹⁰ stands for Lys, the latter are linked to one another via a side chain.

The most preferred peptides of general formula I are

Lys-Ile-Ser-Val-Ser-Tyr-Asp-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25

whereby the Lys in 1-position is linked with the Asp in 13-position via a side chain,

Lys-Ile-Ser-Val-Ser-Tyr-Glu-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25

whereby the Lys in 1-position is linked with the Asp in 13-position and Glu in 7-position is linked with Lys in 19-position via a side chain, and

Lys-Ile-Ser-Val-Ser-Tyr-Glu-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25

whereby the Lys in 1-position is linked with the Asp in 13-position by a β-turn mimetic agent, and the Glu in 7-position is linked with Lys in 19-position via a side chain.

The peptides according to the invention are used as pharmaceutical active ingredients and can be administered alone or in the form of a pharmaceutical composition, which contains one or more peptides of general formula I, together with pharmaceutically suitable solutions and vehicles. The peptides according to the invention can be administered intravenously, subcutaneously, orally or transdermally alone, as a mixture or as a composition together with pharmaceutically suitable solutions and vehicles.

The peptides according to the invention and their compositions and mixtures can be used for the production of a pharmaceutical agent for treating thromboses, unstable angina, arteriosclerosis, prevention of a re-occlusion of vessels after PTCA/PTA or after thrombolysis for treating a myocardial infarction or for preventing blood clotting in the case of hemodialysis. The pharmaceutical active ingredients, compositions or mixtures as well as their uses are also the subject of this invention.

Suitable compositions can also be produced according to processes that are known in the art, whereby all solutions, vehicles and additives that can be used for a formulation of peptides in pharmaceutics can be used (Remington's Pharmaceutical Science, 15th Ed. Mack Publishing Company, East Pennsylvania, 1980).

For therapeutic use, various doses are suitable. The dose that can be administered thus depends on the respective peptide, the individual, the type of administration (intravenous, subcutaneous, oral, transdermal) and on the severity of the disease that is to be treated.

### DESCRIPTION OF THE FIGURES

FIG. 1 shows the extension of the APTT by the inhibitors triabin and hirudin.

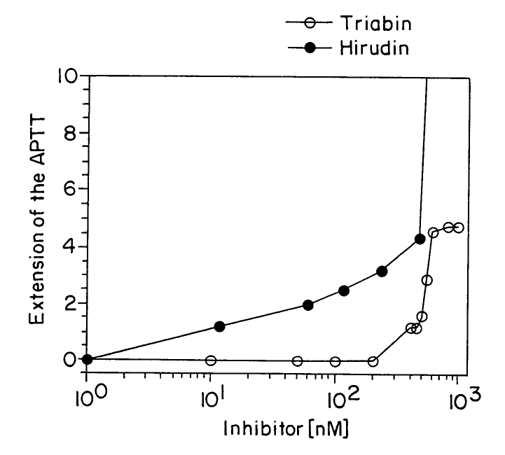

### DESCRIPTION OF THE ABBREVIATIONS

Ala=alanine DMSO=dimethyl sulfoxide

Val=valine DCM=dichloromethane

Leu=leucine DPPF=bis(diphenylphosphino)ferrocene

Ile=isoleucine DMF=dimethylformamide

Pro=proline DIPEA=diisopropylethylamine

Phe=phenylalanine TBTU=benzotriazolyl-tetramethyl-uronium hexafluoroborate

Trp=tryptophan

Met=methionine HOBT=1-hydroxybenzotriazole

Gly=glycine TF4=trifluoroacetic acid

Ser=serine

Thr=threonine

Cys=cysteine

Tyr=tyrosine

Asn=asparagine

Gln=glutamine

Asp=asparaginic acid

Glu=glutamic acid

Lys=lysine

Arg=arginine

His=histidine

Orn=ornithine

Cit=citrulline

β-Ala=β-alanine

homo-Cys=homo-cysteine

homo-Ser=homoserine

Gaba=γ-aminobutyric acid

Can=canavanine

β-CN-Ala=β-cyanoalanine

OH-Pro=hydroxyproline

OH-Lys=hydroxylysine

N-Met-Lys=N-methyllysine

Met-His=methylhistidine

The following examples explain the preliminary examinations and the production of the peptides according to the invention without limiting the latter to the examples.

### EXAMPLE 1

1. Determination of the Crystal Structure of a Complex that Consists of Thrombin and Triabin

Purified triabin and thrombin were added together in 20 mmol of sodium acetate, 25 mmol of sodium chloride, pH 5.5. Crystals of the complex of triabin and thrombin formed in a hanging drop, which contained 50 mmol of sodium acetate, pH 4.7, 100 mmol of ammonium sulfate, 0.01% sodium nitrite and 8% PEG 4000. The structure of the crystals was determined by means of x-ray analysis. The amino acids, which form interactions with thrombin, were determined from these structural data. It has been shown that these amino acids are found in areas that form a β-folded-sheet structure.

The partial sequences of triabin, which bind to thrombin, read:

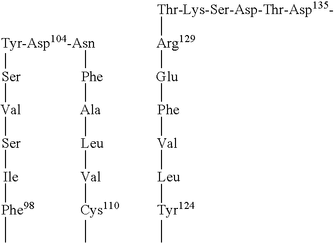

By specific exchange of some amino acids and linkage of the sequences, a peptide was set forth in which the amino acids are spaced at intervals that are similar to the partial sequences of triabin that bind to thrombin. In particular, the phenylalanine in triabin at 98-position was exchanged for lysine, which now represents the first amino acid in the peptide. Cysteine 110 in the triabin was replaced by asparaginic acid in the peptide. The carboxyl side group of the asparaginic acid is linked to the amino side group of lysine (1-position in the peptide), so that a cyclic connection is produced. The asparaginic acid is connected to the second partial sequence of triabin (124-135), which binds to thrombin. The peptide has the following sequence:

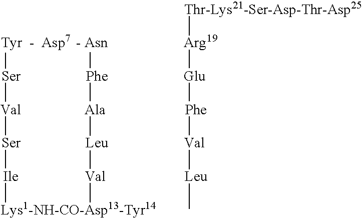

First peptides that contain these areas were set forth. Copying the three-dimensional structure of the original areas was important in the development of the peptides according to the invention. In particular, the β-folded-sheet structure of the area of amino acids 98-103 (named chain 1), amino acids 105-110 (chain 2) and amino acids 124-135 (chain 3) must be sterically stabilized. This could be achieved by different modifications of the original areas according to the following batches.

2. Stabilization of the Peptides

A. Stabilization of Chains 1 and 2

The stabilization was carried out either by

1. Exchange of the amino acids phenylalanine in triabin in 98-position and/or cysteine in 110-position for amino acids, which allow a linkage via the side chain (e.g., Lys-Asp, Cys-Cys, ornithine-Glu)

or by

2. Exchange of Cys110 for Asp and linkage of Phe98 and this Asp by a “β-turn mimetic agent.” The structures of β-turn mimetic agents and their use are described in detail in U. Egner et al. (1997) Pesticide Science, in Press.

B. The Stabilization of Chains 2 and 3

The stabilization was carried out either by

1. Exchange of the L-Tyr in 124-position for a D-Tyr (configuration isomer) and linkage of amino acids in 110-position (in the triabin Cys) with that in 124-position (in the triabin Tyr) by a peptide bond

or by

2. Exchange of the L-Tyr in 124-position for a D-Tyr and linkage of the amino acid in 110-position (in the triabin Cys) with the D-Tyr via a “β-turn mimetic agent” as described in U. Egner et al. (1997) Pesticide Science, in Press.

C. Additional Stabilization

Additional stabilization of chains 2 and 3 can be achieved by linkage of the side chains of amino acids 104 and 129. This can be carried out by, e.g., exchange of Asp 104 by Glu and exchange of Arg 129 by Lys and linkage of this Glu with the Lys by the side chains.

By combination of the batches that are described for stabilization of the structure, the various peptides according to the invention can be synthesized.

### EXAMPLE 2

Production of the Peptides

A peptide was synthesized starting from the C-terminus (Asp25) according to the Merrifield solid-phase protein method with the aid of a peptide-synthesis machine with use of the Fmoc chemistry. For cyclization, the side chains of lysine 1 and of asparaginic acid 13 must be selectively deprived of protection; therefore N-a-1-(4,4-dimethyl-2,6-dioxocyclohex-1-ylidine)ethyl-N-e-Fmoc-L-lysine (Dde-Lys) was used for 1-position, while Boc-lysine (Fmoc) was used for the lysine in 21-position. Asp (O-All) was used for Asp in 13-position and Asp (O-tBu)-Asp(Fmoc) was used for the others. The synthesis was structured into the following steps:

1. Synthesis of the Peptide

The synthesis was carried out according to standard methods in a peptide-synthesis machine of Applied Biosystems.

Sequence: Lys-Ile-Ser-Val-Ser-Tyr-Asp-Asn-Phe-Ala-Leu-Val-Asp-D-Tyr-Leu-Val-Phe-Glu-Arg-Thr-Lys-Ser-Asp-Thr-Asp

2. Cleavage of the Dde and O-All Groups

In addition, the resin was washed twice with DMSO/DCM (1:1) and allowed to steep in this solution for 30 minutes. Then, palladium (0.1 mol/mol of peptide) and DPPF (0.1 mol/mol of peptide) and acetic acid (10-fold excess) were added. Sn(Bu)₃H was added in 5 portions (a total of 5-fold excess) within 10 minutes. The mixture was stirred for 20 minutes, then suctioned off and washed with DMSO/DCM and DCM.

3. Cyclization

The resin was pre-steeped in DMF for 30 minutes, then DIPEA (8-fold excess), TBTU (2-fold excess) and HOBT (2-fold) were added and stirred overnight. The resin was suctioned off and washed with DMF and ether.

4. Cleavage of Resin and Cleavage of the Residual Protective Groups

Phenol, ethyldithiol, thioanisole, H₂O and TFA were added to the resin, and the reaction mixture was stirred for 4 hours at 37° C. The peptide was precipitated with t-butylether, centrifuged off and dried under nitrogen.

The subsequent sample applications show the use of the peptides according to the invention in comparison to the known proteins triabin and hirudin without limiting the use of the compounds according to the invention to these examples.

Sample Application 1

Action of Triabin and Hirudin on Blood Clotting

The action of triabin and hirudin on blood clotting was measured by determining the activated partial thromboplastin time (APTT). 100 μl of human citrate plasma, 10 μl of inhibitor (triabin or hirudin) and 100 μl of APTT reagent (pathromtin from the Behring Company) were incubated for 3 minutes at 37° C. After 100 μl of a 25 mmol CaCl₂ solution was added, the time until clots formed was measured. The measuring was carried out in a fibrometer of the Sarstedt Company. The results are indicated in an extension of the clotting time, which was measured without adding inhibitor.

Sample Application 2

The action of the peptides according to the invention on the blood clotting was also measured according to the same method (APTT) as described under sample application 1. As an inhibitor, a solution of the peptide, mentioned in Example 2, was added in a concentration of 0.1-100 μmol. Then, the extension of the clotting time was measured as described above.

Sample Application 3

Measurement of the Fibrinogen Cleavage

Microtiter plates were coated first with bovine serum albumin. Then, 100 μl of triabin solution (0.1-10 μmol/l 10 mmol of NaH₂PO₄, pH 7.4) was added to the microtiter plate, and 100 μl of reaction buffer (20 mmol of HEPES, 0.15 M of NaCl, pH 7.4), 20 μl of CaCl₂ solution (20 mmol of CaCl₂ in H₂O) and 20 μl of thrombin solution (0.012 IU) were pipetted into it. After an incubation at 37° C. for 2 minutes, 100 μl of fibrinogen (10 mg in 2 ml of reaction buffer) was added and incubated for 40 minutes at 37° C. Then, the extinction was measured at 405 nm. As a control value (100% thrombin activity), 100 μl of reaction buffer was used in a batch instead of the triabin solution. The measured values with triabin were related to these values and expressed as % of inhibition.

1. Peptides of general formula I

| Y¹-X¹-Ser-X²-Ser-X³-X⁴-Asn-Phe-X⁵-X⁶-X⁷-Y²-D-Tyr-X⁸-Val-X⁹-Glu-X¹⁰- | (I) |
|---|---|
| 1 2 3 4 5 6 7 8 9 10 11 12 13 d 14 15 16 17 18 19 | |
| X¹¹-X¹²-Ser-X¹³-X¹⁴-Asp, | |
| 20 21 22 23 24 25 | |

in which
Y¹ is Phe, Lys, Cys and Orn, and
Y² is Asp, Cys and Glu, and Y¹ also has the meaning of Y², and Y² has the meaning of Y¹, whereby Y¹ and Y² are linked to one another via a side chain or a β-turn mimetic agent, and
X¹⁻¹⁴ represents any amino acid, which can be connected to one another via side chains.

2. Peptides of general formula I, according to claim 1, in which
Y¹ is Phe, Lys, Cys and Orn, and
Y² is Asp, Cys and Glu, and Y¹ also has the meaning of Y², and Y² has the meaning of Y¹, whereby Y¹ and Y² are linked to one another via a side chain or a β-turn mimetic agent, and
X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn, Cit, β-Ala, homo-Cys, homo-Ser, Gaba, Can, β-CN-Ala, OH-Pro, OH-Lys, N-Met-Lys, Met-His, desmosine and djenkolic acid, which can be connected to one another via side chains.

3. Peptides of general formula I, according to claims 1-2, in which
Y¹ is Phe, Lys, Cys and Orn, and
Y² is Asp, Cys and Glu, and Y¹ also has the meaning of Y², and Y² has the meaning of Y¹, whereby Y¹ and Y² are linked to one another via a side chain or a β-turn mimetic agent, and
X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn, and Cit, which can be connected to one another via side chains.

4. Peptides of general formula I, according to claims 1-3, in which
Y¹ is Lys, Cys and Orn, and
Y² is Asp, Cys, and Glu, and Y¹ also has the meaning of Y², and Y² has the meaning of Y¹, whereby Y¹ and Y² are linked to one another via a side chain, and
X⁶ and X⁸ are Leu,
X⁷ is Val and
X¹⁻⁵ and X⁹⁻¹⁴ are Ala, Val, Leu, Ile, Pro, Phe, Trp, Met, Gly, Ser, Thr, Cys, Tyr, Asn, Gln, Asp, Glu, Lys, Arg, His, Orn and Cit, whereby if
X⁴ stands for Glu, and X¹⁰ stands for Lys, the latter are linked to one another via a side chain.

5. Peptides of general formula I, according to claims 1-4, in which
Y¹ is Lys, and
Y² is Asp, and Y¹ also has the meaning of Y², and Y² has the meaning of Y¹, whereby Y¹ and Y² are linked to one another via a β-turn mimetic agent, and
X¹⁻¹⁴ is Ala, Val, Leu, Ile, Pro, Phe, Trp, Met, Gly, Ser, Thr, Cys, Tyr, Asn, Gln, Asp, Glu, Lys, Arg, His, Orn and Cit, whereby if X⁴ stands for Glu and X¹⁰ stands for Lys, the latter are linked to one another via a side chain.

6. Compounds of general formula I, according to claims 1-5, in which
Y¹ is Lys, and
Y² is Asp, and Y¹ also has the meaning of Y², and Y² has the meaning of Y¹, whereby Y¹ and Y² are linked to one another via a β-turn mimetic agent, and
X⁶ and X⁸ are Leu,
X⁷ is Val,
X¹⁻⁵ and X⁹⁻¹⁴ are Ala, Val, Leu, Ile, Pro, Phe, Trp, Met, Gly, Ser, Thr, Cys, Tyr, Asn, Gln, Asp, Glu, Lys, Arg, His, Orn and Cit, whereby if
X⁴ stands for Glu, and X¹⁰ stands for Lys, the latter are linked to one another via a side chain.

7. Peptides of general formula I, according to claims 1-6, the structures
Lys-Ile-Ser-Val-Ser-Tyr-Asp-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25
whereby the Lys in 1-position is linked with the Asp in 13-position via a side chain,
Lys-Ile-Ser-Val-Ser-Tyr-Glu-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25
whereby the Lys in 1-position is linked with the Asp in 13-position, and Glu in 7-position is linked with Lys in 19-position via side chain, and
Lys-Ile-Ser-Val-Ser-Tyr-Glu-Asn-Phe-Ala-Leu-Val-Asp-D-Tyr-
1 2 3 4 5 6 7 8 9 10 11 12 13 14
Leu-Val-Phe-Glu-Arg-Thr-Lys-Ser-Asp-Thr-Asp,
15 16 17 18 19 20 21 22 23 24 25
whereby the Lys in 1-position is linked with the Asp in 13-position by a β-turn mimetic agent, and the Glu in 7-position is linked with Lys in 19-position via a side chain.

8. A peptide of general formula I, according to one of claims 1-7, as a pharmaceutical active ingredient.

9. A pharmaceutical composition that contains one or more peptides of general formula I, according to claims 1-7, together with pharmaceutically suitable solutions and vehicles.

10. A composition that can be administered intravenously, subcutaneously, orally or transdermally that contains one or more peptides of general formula I, according to claims 1-7, together with pharmaceutically suitable solutions, vehicles and additives.

11. Use of the peptides according to claims 1-8 and their compositions according to claims 9 and 10 or mixtures thereof, for the production of a pharmaceutical agent for treating thromboses, unstable angina, arteriosclerosis, prevention of a re-occlusion of vessels after PTCA/PTA or after thrombolysis for treating a myocardial infarction or for preventing blood clotting in the case of hemodialysis.


SEQUENCE LISTING (3 sequences)

SEQ ID NO: 1 - protein, 13 aa, Artificial Sequence
  Description of Artificial Sequence Synthetic peptide
FISVSYDNFALVC

SEQ ID NO: 2 - protein, 12 aa, Artificial Sequence
  Description of Artificial Sequence Synthetic peptide
YLVFERTKSDTD

SEQ ID NO: 3 - protein, 25 aa, Artificial Sequence
  Description of Artificial Sequence Synthetic peptide
KISVSYDNFALVDYLVFERTKSDTD